(19) United States
(12) Patent Application Publication
(10) Pub. No.: US 2025/0221922 A1
(43) Pub. Date: Jul. 10, 2025

(54) ORAL CARE COMPOSITIONS CONTAINING RHAMNOLIPIDS

(71) Applicant: Stepan Company, Northbrook, IL (US)

(72) Inventors: Kelly Strout, Lake Zurich, IL (US); Alexander Nikoloff, Wheeling, IL (US); Thach Bao Tran, Des Plaines, IL (US); John MacKinnon, Northbrook, IL (US); Jesse Jefferis, Lincolnshire, IL (US); Cyril Bajracharya, Arlington Heights, IL (US)

(21) Appl. No.: 19/095,301

(22) Filed: Mar. 31, 2025

Related U.S. Application Data
(63) Continuation of application No. PCT/US2023/076048, filed on Oct. 5, 2023.
(60) Provisional application No. 63/413,379, filed on Oct. 5, 2022.

(51) Int. Cl. A61K 8/99; A61Q 11/00

(52) U.S. Cl. CPC A61K 8/99; A61Q 11/00; A61K 2800/74

(57) ABSTRACT

Oral care compositions that contain a particular mixture of mono-rhamnolipids and di-rhamnolipids are disclosed. The compositions have antimicrobial efficacy against oral microbes, such as P. gingivalis, S. mutans, and C. rectus. Also disclosed is a method for treating or preventing gingivitis in a subject. Also disclosed is a method for preventing growth of microorganisms as biofilm and/or reducing a population of microorganisms present as biofilm in the oral cavity of a subject.

### BACKGROUND OF THE INVENTION

The present technology, in general, relates to oral care compositions that comprise biosurfactants, such as rhamnolipids. More particularly, the present technology relates to oral care compositions comprising rhamnolipids that have efficacy against oral relevant microbes, including biofilm-forming bacteria.

There has been a recent trend to formulate products with ingredients that are based on renewable raw materials. Such ingredients are considered “green” or “natural”, since they are derived from renewable and/or sustainable sources. As a result, they are more environmentally friendly than ingredients derived from fossil fuels or other non-renewable sources. An ingredient having a high Biorenewable Carbon Index (BCI), such as greater than 80, indicates that the ingredient contains carbons that are derived primarily from plant, animal or marine-based sources.

Rhamnolipids are interface-active glycolipids produced by various bacterial species and are an example of a “green” ingredient, since they can be prepared by means of fermentation based on renewable raw materials. It would be desirable to have oral care compositions that include actives, such as rhamnolipids, that are derived from renewable sources, but that also have effective antimicrobial properties to enable the oral care compositions to be used in the reduction of oral care relevant microbes (enveloped and non-enveloped) and treatment/prevention of associated biofilms.

Applicants have determined that the use of rhamnolipids in oral care compositions can meet the above objectives while also advancing UN Sustainability Goals (“SDG”). The oral care compositions of the present technology, and related methods of use thereof, can contribute to better health and well-being by delivering efficacy against oral care relevant microbes and biofilms using a rhamnolipid active. Advantageously, the rhamnolipids are bio-based, renewably sourced actives obtained from a bacterial fermentation process that generates biodegradable waste products that are less impactful on the environment. These benefits further SDG #3 (Good Health and Well-being) and SDG #12 (Responsible Consumption and Production).

### BRIEF SUMMARY OF THE INVENTION

In one aspect, the present technology provides an oral care composition comprising a mixture of purified rhamnolipids in an amount of about 0.025 to about 99% by weight, based on the total weight of the composition, wherein the mixture of rhamnolipids comprises mono-rhamnolipids and di-rhamnolipids in a weight ratio of 10:90 to 90:10 mono-rhamnolipids:di-rhamnolipids, alternatively 15:85 to 90:10 mono-rhamnolipids:di-rhamnolipids, alternatively 20:80 to 90:10 mono-rhamnolipids:di-rhamnolipids, alternatively 30:70 to 90:10 mono-rhamnolipids:di-rhamnolipids, alternatively 40:60 to 90:10 mono-rhamnolipids:di-rhamnolipids, alternatively 30:70 to 80:20 mono-rhamnolipids:di-rhamnolipids, alternatively 30:70 to 70:30 mono-rhamnolipids:di-rhamnolipids, alternatively 30:70 to 60:40 mono-rhamnolipids:di-rhamnolipids, alternatively 30:70 to 50:50 mono-rhamnolipids:di-rhamnolipids, alternatively 30:70 to 45:55 mono-rhamnolipids:di-rhamnolipids, alternatively 40:60 to 60:40 mono-rhamnolipids:di-rhamnolipids, alternatively 40:60 to 50:50 mono-rhamnolipids:di-rhamnolipids, alternatively 40:60 to 45:55 mono-rhamnolipids:di-rhamnolipids, alternatively 40:60 to 48:52 mono-rhamnolipids:di-rhamnolipids, as measured by high-performance liquid chromatography (HPLC).

In a related aspect, the present technology provides an oral care composition comprising a mixture of purified rhamnolipids in an amount of 0.025% to 99% by weight, based on the total weight of the composition, wherein the mixture of rhamnolipids comprises at least 10% mono-rhamnolipids (as weight % of total rhamnolipids), alternatively at least 15% mono-rhamnolipids, alternatively at least 20% mono-rhamnolipids, alternatively at least 25% mono-rhamnolipids, alternatively at least 30% mono-rhamnolipids, alternatively at least 35% mono-rhamnolipids, alternatively at least 40% mono-rhamnolipids, as measured by high-performance liquid chromatography (HPLC).

In another aspect, the present technology provides an oral care composition comprising a mixture of purified rhamnolipids in an amount of 0.025% to 99% by weight, based on the total weight of the composition, wherein the mixture of rhamnolipids comprises mono-rhamnolipids and di-rhamnolipids in a weight ratio of about 10:90 to about 47:53, alternatively about 20:80 to about 47:53, alternatively about 30:70 to about 47:53, alternatively about 40:60 to about 47:53, alternatively about 40:60 to about 45:55, alternatively from 43:57 to 45:55, an amount of Rha-C10-C10 mono-rhamnolipid salt of about 5% to about 38% by weight, alternatively 29% to about 40%, alternatively about 29% to 37.5%, based on the total weight of the rhamnolipids in the mixture of rhamnolipids, and an amount of RhaRha-C10-C10 di-rhamnolipid salt of about 34% to about 80% by weight, 34% to about 45% by weight, alternatively about 36% to about 45% by weight based on the total weight of the rhamnolipids as measured by high-performance liquid chromatography (HPLC).

In some embodiments, the purified rhamnolipid mixture may comprise an amount of Rha-C10-C10 mono-rhamnolipid of about 5% to about 38% by weight, based on the total weight of the rhamnolipids, and an amount of RhaRha-C10-C10 di-rhamnolipid of about 34% to about 80% by weight, based on the total weight the rhamnolipids as measured by high-performance liquid chromatography (HPLC).

In some embodiments, the purified mixture of rhamnolipids comprises mono-rhamnolipids and di-rhamnolipids in a weight ratio of 40:60 to 45:55, an amount of Rha-C10-C10 mono-rhamnolipid of about 29% to 37.5% by weight, and an amount of RhaRha-C10-C10 di-rhamnolipid of about 36% to 45% by weight, based on the total weight of the rhamnolipids as measured by high-performance liquid chromatography (HPLC).

A further aspect of the present technology provides a method for treating or preventing gingivitis in a subject afflicted with or susceptible to gingivitis comprising administering to the subject an effective amount of a composition comprising a mixture of rhamnolipids, e.g. as described above, thereby treating or preventing gingivitis in the subject.

A further aspect of the present technology provides a method for treating or preventing gingivitis in a subject afflicted with or susceptible to gingivitis comprising administering to the subject an effective amount of a composition comprising a mixture of purified rhamnolipids, thereby treating or preventing gingivitis in the subject, wherein the mixture of rhamnolipids comprises mono-rhamnolipids and di-rhamnolipids in a weight ratio of 10:90 to 90:10 mono-rhamnolipids:di-rhamnolipids, alternatively 15:85 to 90:10 mono-rhamnolipids:di-rhamnolipids, alternatively 20:80 to 90:10 mono-rhamnolipids:di-rhamnolipids, alternatively 30:70 to 90:10 mono-rhamnolipids:di-rhamnolipids, alternatively 40:60 to 90:10 mono-rhamnolipids:di-rhamnolipids, alternatively 30:70 to 80:20 mono-rhamnolipids:di-rhamnolipids, alternatively 30:70 to 70:30 mono-rhamnolipids:di-rhamnolipids, alternatively 30:70 to 60:40 mono-rhamnolipids:di-rhamnolipids, alternatively 30:70 to 50:50 mono-rhamnolipids:di-rhamnolipids, alternatively 30:70 to 45:55 mono-rhamnolipids:di-rhamnolipids, alternatively 40:60 to 60:40 mono-rhamnolipids:di-rhamnolipids, alternatively 40:60 to 50:50 mono-rhamnolipids:di-rhamnolipids, alternatively 40:60 to 45:55 mono-rhamnolipids:di-rhamnolipids, as measured by high-performance liquid chromatography (HPLC).

In some embodiments, the present technology provides a method for treating or preventing gingivitis in a subject afflicted with or susceptible to gingivitis, as described above, in which the purified rhamnolipid mixture may comprise an amount of Rha-C10-C10 mono-rhamnolipid of about 5% to about 38% by weight, based on the total weight of the rhamnolipids, and an amount of RhaRha-C10-C10 di-rhamnolipid of about 34% to about 80% by weight, based on the total weight the rhamnolipids as measured by high-performance liquid chromatography (HPLC).

In some embodiments, the present technology provides a method for treating or preventing gingivitis in a subject afflicted with or susceptible to gingivitis, as described above, in which the mixture of purified rhamnolipids comprises mono-rhamnolipids and di-rhamnolipids in a weight ratio of 40:60 to 45:55, an amount of Rha-C10-C10 mono-rhamnolipid of about 29% to 37.5% by weight, and an amount of RhaRha-C10-C10 di-rhamnolipid of about 36% to 45% by weight, based on the total weight of the rhamnolipids as measured by high-performance liquid chromatography (HPLC).

A further aspect of the present technology provides a method for preventing growth of oral bacteria and/or reducing a population of oral bacteria in a subject, in either planktonic or biofilm form, comprising administering to a subject an effective amount of a composition, e.g. as described above, comprising a mixture of rhamnolipids to provide inhibitory and/or cidal effects on target microbes.

A further aspect of the present technology provides a method for preventing growth of oral bacteria and/or reducing a population of oral bacteria in a subject, in either planktonic or biofilm form, comprising administering to a subject an effective amount of a composition comprising a mixture of rhamnolipids to provide inhibitory and/or cidal effects on target microbes, wherein the mixture of rhamnolipids comprises mono-rhamnolipids and di-rhamnolipids in a weight ratio of about 10:90 to about 47:53, alternatively about 20:80 to about 47:53, alternatively about 30:70 to about 47:53, alternatively about 40:60 to about 47:53, alternatively about 40:60 to about 45:55, alternatively from 43:57 to 45:55 mono-rhamnolipids:di-rhamnolipids, as measured by high-performance liquid chromatography (HPLC).

In some embodiments, the present technology provides a method for preventing growth of oral bacteria and/or reducing a population of oral bacteria in a subject, in either planktonic or biofilm form, as described above, in which the rhamnolipid mixture may comprise an amount of Rha-C10-C10 mono-rhamnolipid of about 5% to about 38% by weight, based on the total weight of the rhamnolipids, and an amount of RhaRha-C10-C10 di-rhamnolipid of about 34% to about 80% by weight, based on the total weight of the rhamnolipids as measured by high-performance liquid chromatography (HPLC).

In some embodiments, the present technology provides a method for preventing growth of oral bacteria and/or reducing a population of oral bacteria in a subject, in either planktonic or biofilm form, as described above, in which the mixture of rhamnolipids comprises mono-rhamnolipids and di-rhamnolipids in a weight ratio of 40:60 to 45:55, an amount of Rha-C10-C10 mono-rhamnolipid of about 29% to 37.5% by weight, and an amount of RhaRha-C10-C10 di-rhamnolipid of about 36% to 45% by weight, based on the total weight of the rhamnolipids as measured by high-performance liquid chromatography (HPLC).

In another aspect of the present technology, the purified rhamnolipid compositions disclosed herein are associated with certain biological activities. One such property is the capacity to reduce viability and the biological activity (e.g., growth) of Porphyromonas gingivalis. For example, in one aspect, the rhamnolipid compositions disclosed herein display a characteristic Minimum Inhibitory Concentration (MIC) for Porphyromonas gingivalis, determined as set forth in Example 1 (P. gingivalis Inhibitory and Bactericidal Efficacy Testing). In this aspect, the rhamnolipid compositions disclosed herein display a MIC for P. gingivalis, determined in accordance with the method set forth in Example 1 (P. gingivalis Inhibitory and Bactericidal Efficacy Testing), of from about 21 ppm to about 106 ppm, from about 25 ppm to about 106 ppm, from about 30 ppm to about 106 ppm, from about 35 ppm to about 106 ppm, from about 40 ppm to about 106 ppm, or from about 41 ppm to about 106 ppm.

In a still further aspect of the present technology, the rhamnolipid compositions disclosed herein display a characteristic Minimum Bacteriocidal Concentration (MBC) for Porphyromonas gingivalis, determined as set forth in Example 1 (P. gingivalis Inhibitory and Bactericidal Efficacy Testing). In this aspect, the rhamnolipid compositions disclosed herein display a MBC for P. gingivalis, determined in accordance with the method set forth in Example 1 (P. gingivalis Inhibitory And Bactericidal Efficacy Testing), of from about 62 ppm to about 296 ppm, from about 70 ppm to about 296 ppm, from about 80 ppm to about 296 ppm, from about 90 ppm to about 296 ppm, from about 100 ppm to about 296 ppm, from about 110 ppm to about 296 ppm, from about 120 ppm to about 296 ppm, or from about 123 ppm to about 296 ppm.

In still another aspect, the rhamnolipid compositions disclosed herein display a characteristic Minimum Inhibitory Concentration (MIC) for Streptococcus mutans, determined as set forth in Example 2 (S. mutans Inhibitory and Bactericidal Efficacy Testing). In this aspect, the rhamnolipid compositions disclosed herein display a MIC for S. mutans, determined in accordance with the method set forth in Example 2 (S. mutans Inhibitory and Bactericidal Efficacy Testing), of from about 62 ppm to about 889 ppm, from about 70 ppm to about 889 ppm, from about 80 ppm to about 889 ppm, from about 90 ppm to about 889 ppm, from about 100 ppm to about 889 ppm, from about 110 ppm to about 889 ppm, from about 120 ppm to about 889 ppm, from about 123 ppm to about 889 ppm.

In a still further aspect of the present technology, the rhamnolipid compositions disclosed herein display a characteristic Minimum Bactericidal Concentration (MBC) for Streptococcus mutans, determined as set forth in Example 2 (S. mutans Inhibitory and Bactericidal Efficacy Testing). In this aspect, the rhamnolipid compositions disclosed herein display a MBC for S. mutans, determined in accordance with the method set forth in Example 2 (S. mutans Inhibitory and Bactericidal Efficacy Testing), of from about 556 ppm to about 2666 ppm, from about 600 ppm to about 2666 ppm, from about 700 ppm to about 2666 ppm, from about 800 ppm to about 2666 ppm, from about 900 ppm to about 2666 ppm, from about 1000 ppm to about 2666 ppm, from about 1100 ppm to about 2666 ppm, or from about 1111 ppm to about 2666 ppm.

In still another aspect, the rhamnolipid compositions disclosed herein display a characteristic Minimum Inhibitory Concentration (MIC) for Campylobacter rectus, determined as set forth in Example 3 (C. rectus Inhibitory and Bactericidal Efficacy Testing). In this aspect, the rhamnolipid compositions disclosed herein display a MIC for C. rectus, determined in accordance with the method set forth in Example 3 (C. rectus Inhibitory and Bactericidal Efficacy Testing), of from about 21 ppm to about 98 ppm, from about 25 ppm to about 98 ppm, from about 30 ppm to about 98 ppm, from about 35 ppm to about 98 ppm, from about 40 ppm to about 98 ppm, or from about 41 ppm to about 98 ppm.

In still another aspect, the rhamnolipid compositions disclosed herein display a characteristic Minimum Bactericidal Concentration (MBC) for Campylobacter rectus, determined as set forth in Example 3 (C. rectus Inhibitory and Bactericidal Efficacy Testing). In this aspect, the rhamnolipid compositions disclosed herein display a MBC for C. rectus, determined in accordance with the method set forth in Example 3 (C. rectus Inhibitory and Bactericidal Efficacy Testing), of from about 21 ppm to about 98 ppm, from about 25 ppm to about 98 ppm, from about 30 ppm to about 98 ppm, from about 35 ppm to about 98 ppm, from about 40 ppm to about 98 ppm, or from about 41 ppm to about 98 ppm.

In another aspect, the rhamnolipid compositions disclosed herein display a characteristic Log 10 reduction for P. gingivalis, determined as set forth in Example 7 (with rhamnolipid present in an amount of 0.1% by weight of the composition, and a 30 second contact time), of from about 3 to about 6, from about 3.5 to about 6, from about 4 to about 6, from about 4.5 to about 6, from about 5 to about 6, or from about 5.5 to about 6.

In another aspect, the rhamnolipid compositions disclosed herein display a characteristic Log 10 reduction for P. gingivalis, determined as set forth in Example 7 (with rhamnolipid present in an amount of 0.05% by weight of the composition, and a 30 second contact time), of from about 2.5 to about 6, from about 3 to about 6, from about 3.5 to about 6, from about 4 to about 6, from about 4.5 to about 6, from about 5 to about 6, or from about 5.5 to about 6.

In another aspect, the rhamnolipid compositions disclosed herein display a characteristic Log 10 reduction for P. gingivalis, determined as set forth in Example 7 (with rhamnolipid present in an amount of 0.025% by weight of the composition, and a 30 second contact time), of from about 2 to about 6, of from about 2.5 to about 6, of from about 3 to about 6, from about 3.5 to about 6, from about 4 to about 6, from about 4.5 to about 6, from about 5 to about 6, or from about 5.5 to about 6.

### DETAILED DESCRIPTION OF THE INVENTION

### Definitions

“Biorenewable Carbon Index” (BCI) refers to a calculation of the percent carbon derived from a biorenewable resource, and is calculated based on the number of biorenewable carbons divided by the total number of carbons in the entire molecule.

“Biorenewable” is defined herein as originating from animal, plant, or marine material.

As used herein, “effective amount” refers to an amount of an active ingredient or composition that, when administered to a subject, is capable of preventing the identified microorganism(s) and/or reducing a population of the identified microorganism(s) in the oral cavity of a subject. The actual amount may vary depending on a number of factors, including, but not limited to, the titer of the microorganism population, the presence and amount of biofilm, the age and health status of the subject, and the form of administration.

As defined herein, a “rhamnolipid” is a glycolipid that has a lipid portion that includes one or more, typically linear, saturated or unsaturated β-hydroxy-carboxylic acid moieties and a saccharide portion of one or more units of rhamnose.

As defined herein, unless otherwise specified, “Log₁₀ Reduction” is calculated using the following formula: Log₁₀ Reduction=Log₁₀(Untreated)−Log₁₀(Treated).

The saccharide portion and the lipid portion are linked via a β-glycosidic bond between the 1-OH group of a rhamnose moiety of the saccharide portion and the 3-OH group of a β-hydroxy-carboxylic acid of the lipid portion. Thus, the carboxylic acid of one carboxylic acid moiety defines the end of the rhamnolipid. Where more than one rhamnose-moiety is included in a rhamnolipid, each of the rhamnose moieties not linked to the lipid portion is linked to another rhamnose moiety via a 1,40-glycosidic bond. In embodiments where two or more β-hydroxy-carboxylic acids are present in a rhamnolipid, the β-hydroxy-carboxylic acid moieties are selected independently from each other. β-hydroxy carboxylic acid moieties may in some embodiments be identical. In some embodiments, they are different from each other.

The present technology generally relates to oral care compositions that comprise a particular mixture of rhamnolipids. Although rhamnolipids have been found to demonstrate antimicrobial activity, the particular mixture of rhamnolipids described herein provides greater efficacy than other rhamnolipid mixtures known in the art against anaerobic bacteria within oral care applications. The rhamnolipids in the mixture of rhamnolipids of the present technology may have the following structure (I):

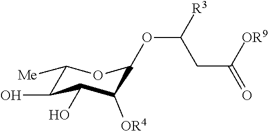

In this formula, R⁹ is a hydrogen atom (H) or an aliphatic group that has a main chain of one to about 46, such as one to about 42, one to about 40, one to about 38, one to about 36, one to about 34, one to about 30, one to about 28, including e.g. 2, 3, 4, 5, 6, 7, 8, 9, 10, 11, 12, 13, 14, 15, 16, 17, 18, 19, 20, 21, 22, 23, 24, 25, 26, 27 or 28 carbon atoms and one to about three, including two, oxygen atoms. In some embodiments, the main chain of the respective aliphatic group carries a terminal carboxylic acid group and/or an internal ester group. As an illustrative example in this regard, R⁹ may be of the formula —CH(R⁵)—CH₂— COOR⁶. In these illustrative moieties, R⁵ may be an aliphatic moiety with a main chain that has a length from 1 to about 19, such as from 1 to about 17, from 1 to about 15, from 1 to about 13, about 2 to about 13, about 3 to about 13 or about 4 to about 13, including e.g. 2, 3, 4, 5, 6, 7, 8, 9, 10, 11 or 12 carbon atoms. R⁴ in formula (I) is a hydrogen atom (H), or a rhamnopyranosyl moiety. R⁶ is a hydrogen atom.

The term “aliphatic” means, unless otherwise stated, a straight or branched hydrocarbon chain, which may be saturated or mono- or poly-unsaturated and include heteroatoms. The term “heteroatom” as used herein means an atom of any element other than carbon or hydrogen. Herein, an unsaturated aliphatic group contains one or more double bonds (alkenyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to about 5, to about 10, to about 15 or to about 20 carbon atoms. Examples of alkenyl moieties are straight-chain or branched hydrocarbon moieties that contain one or more double bonds. Alkenyl moieties generally contain about two to about twenty carbon atoms and one or more, for instance two, double bonds, such as about two to about ten carbon atoms, and one double bond. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3-dimethylbutyl. Both the main chain as well as the branches may furthermore contain heteroatoms as for instance N, O, S, Se or Si or a carbon atom may be replaced by one of these heteroatoms. An aliphatic moiety may be substituted or unsubstituted with one or more functional groups. Substituents may be any functional group, as for example, but not limited to, amino, amido, carbonyl, carboxyl, hydroxyl, nitro, thio and sulfonyl.

In a more particular embodiment, the rhamnolipids or rhamnolipid salts in said structure has the structure (II):

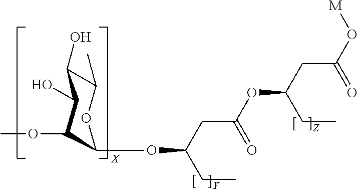

wherein x is 1 or 2; y is 4, 6 or 8; z is 4, 6, or 8; and M is H or a metal, such as alkali metals Li, Na, or K, alkali earth metals Mg or Ca, or transition metals Mn, Fe, Cu, or Zn. In the cases of the alkali earth and transition metals, multiple rhamnolipid salt moieties may associate with each metal.

The mixture of rhamnolipids comprises a mixture of mono-rhamnolipids and di-rhamnolipids.

In some embodiments, the mono-rhamnolipids may be present in an amount of about 10% to 90%, alternatively about 15% to 90%, alternatively about 20% to about 90%, alternatively about 30% to about 90%, alternatively about 40% to about 90%, alternatively about 10% to about 80%, alternatively about 15% to about 80%, alternatively about 20% to about 80%, alternatively about 30% to about 80%, alternatively about 40% to about 80%, alternatively about 30% to about 70%, alternatively about 30% to about 60%, alternatively about 30% to about 50%, based on the total weight of rhamnolipids and as measured by high-performance liquid chromatography (HPLC).

In some embodiments, the mono-rhamnolipids may be present in an amount of about 10% to about 47%, alternatively about 15% to about 45%, alternatively about 20% to about 45%, alternatively about 30% to about 45%, alternatively about 40% to about 45%, alternatively about 43% to about 45%, 10% to about 48%, alternatively about 15% to about 48%, alternatively about 20% to about 48%, alternatively about 30% to about 48%, alternatively about 40% to about 48%, alternatively about 43% to about 48% alternatively about 10% to about 47%, alternatively about 15% to about 47%, alternatively about 20% to about 47%, alternatively about 30% to about 47%, alternatively about 40% to about 47%, alternatively about 43% to about 47%, based on the total weight of rhamnolipids.

The di-rhamnolipids may be present in an amount of about 52% to about 90% by weight, alternatively about 52% to about 85%, alternatively about 52% to about 80%, alternatively about 52% to about 70%, alternatively about 52% to about 60%, alternatively about 52% to about 57%, alternatively, about 53% to about 90% by weight, alternatively about 53% to about 85%, alternatively about 53% to about 80%, alternatively about 53% to about 70%, alternatively about 53% to about 60%, alternatively about 53% to about 57%, alternatively about 54% to about 85%, alternatively about 55% to about 80%, alternatively about 55% to about 70%, alternatively about 55% to about 60%, alternatively about 55% to about 57% by weight, based on the total weight of rhamnolipids and as measured by high-performance liquid chromatography (HPLC). The ratio of mono-rhamnolipids:di-rhamnolipids can be from about 10:90 to about 48:52, alternatively about 47:53, alternatively about 40:60 to about 45:55. In some embodiments, the ratio of mono-rhamnolipids:di-rhamnolipids can range from 43.5:56.5 to 45:55, alternatively from 43:57 to 45:55 or from 43:57 to 48:52.

The mixture of rhamnolipids preferably comprises mono (where x=1) and di (where x=2) rhamnolipids where y and z are 6 and M is H or Na. The mono-rhamnolipid may be referred to as Rha-C10-C10, with a formula of C₂₆H₄₈O₉. The IUPAC Name is 3-[3-[(2R,3R,4R,5R,6S)-3,4,5-trihydroxy-6-methyloxan-2-yl]oxydecanoyloxy]decanoic acid. The di-rhamnolipid may be referred to as RhaRha-C10-C10, with a formula of C₃₂H₅₈O₁₃. The IUPAC name is 3-[3-[4, 5-dihydroxy-6-methyl-3-(3,4,5-trihydroxy-6-methyloxan-2-yl)oxyoxan-2-yl]oxydecanoyloxy]decanoic acid. Rha-C10-C10 may be present in the mixture in an amount of about 5% to 38%, alternatively about 10% to about 38%, alternatively about 15% to about 37.5%, alternatively about 20% to about 37.5%, alternatively about 25% to about 37.5%, alternatively about 29% to about 37.5%, alternatively about 35% to about 37% by weight based on the total weight of rhamnolipids, as measured by high-performance liquid chromatography (HPLC). RhaRha-C10-C10 may be present in the mixture in an amount of about 34% to about 80%, alternatively about 35% to about 75%, alternatively about 35% to about 65%, alternatively about 35% to about 60%, alternatively about 35% to about 55%, alternatively about 35.5% to about 50%, alternatively about 36% to about 45%, alternatively about 36% to about 38% by weight based on the total weight of rhamnolipids in the mixture of rhamnolipids, as measured by high-performance liquid chromatography (HPLC).

In addition to Rha-C10-C10 and RhaRha-C10-C10, the mixture of rhamnolipids may comprise RhaRha-C10-C12 in an amount of about 5% to about 15%, alternatively about 9% to about 12%, alternatively about 10% to about 12.5% by weight based on the total weight of rhamnolipids, and Rha-C10-C12 in an amount of about 0.2% to about 6% by weight, alternatively about 2% to about 5%, alternatively about 3.5% to about 5% by weight based on the total weight of rhamnolipids, each being measured by high-performance liquid chromatography (HPLC). The mixture of rhamnolipids may also comprise, for example, an amount of RhaRha-C10-C12:1 in an amount of about 0.2% to about 5% by weight, alternatively 1% to about 4% by weight, based on the total weight of rhamnolipids; an amount of RhaRha-C8-C10 in the range of about 0.2% to about 5% by weight, alternatively about 1% to about 4% by weight, based on the total weight of rhamnolipids; an amount of Rha-C8-C10 in the range of about 0.2% to about 5% by weight, alternatively about 1% to about 4% by weight, based on the total weight of rhamnolipids; or any combination thereof, each being measured by high-performance liquid chromatography (HPLC).

The rhamnolipids may be produced from a rhamnolipid-producing microorganism that has the capacity to synthesize/produce rhamnolipids under suitable conditions. Such microorganisms include, but are not limited to, bacteria, particularly bacteria of the phyla Actinobacteria, Firmicutes, and Proteobacteria. The rhamnolipids are naturally derived and therefore have a BCI of 100. In a particular embodiment, the rhamnolipid-producing microorganism for producing the rhamnolipids is Pseudomonas aeruginosa.

Methods of culturing the rhamnolipid-producing bacteria and the production of rhamnolipids from fermentation are known in the art from, for example U.S. Pat. Nos. 11,142,782 and 10,144,943, incorporated herein by reference in their entirety. Methods of purifying the rhamnolipids are also known in the art from, for example, U.S. Pat. Nos. 9,884,883 and 10,829,507, incorporated herein by reference in their entirety. The rhamnolipids in the mixture of rhamnolipids used in the present technology are separated from the fermentation medium, washed, deodorized, decolorized, and neutralized to form the purified rhamnolipid salts.

The amount of rhamnolipid component in the oral care compositions may be in the range of from 0.025% to 99% by weight, alternatively from 0.050% to 99% by weight, alternatively from 0.075% to 99% by weight, alternatively from 0.25% to 99% by weight, alternatively from 0.5% to 99% by weight, alternatively 0.1% to 99% by weight, based on the total weight of the composition.

The oral care compositions of the present technology may be in liquid form, and comprise at least one carrier suitable for oral care to bring the total percentage of the composition to 100% (taking into account any additives that may be present in addition to the rhamnolipids). As will be appreciated by at least those skilled in the art, a variety of carriers, vehicles, diluents, and the like are suitable for use in the practice of the present technology. Thus, it will also be appreciated that the terms “carrier”, “vehicle”, and “diluent” are to be considered non-exhaustive and interchangeable with respect to the present technology and in describing the various formulations, applications, uses, and compositions thereof.

Water is a suitable carrier and can be de-ionized water, hard water, soft water, distilled water, tap water or combinations thereof. Water can be used alone as the carrier, or in combination with other carriers suitable for oral care applications.

The oral care compositions of the present technology can include optional ingredients as known in the art. Such other components or additives can include pH adjustment agents, hydrotropic or other solubilizing agents for obtaining and maintaining a clear single-phase composition, emulsifiers, binding agents, excipients, stabilizing agents, chelating agents, gelling agents, buffering agents, surfactants, lubricants, flavor ingredients, sweetening agents, colorants, vitamins, fluoride sources, preservatives, and other functional ingredients.

In some embodiments, for instance, the oral care composition may include a fluoride source such as stannous fluoride, sodium fluoride, potassium fluoride, sodium monofluorophosphate, potassium monofluorophosphate, ammonium fluoride, ammonium monofluorophosphate, fluoride-containing amines, and the like. The oral care composition may, in some embodiments, further include an abrasive. The abrasive may for example be a silica abrasive, such as a hydrated silica, precipitated silica, or silica gel, an alternative abrasive material such as a phosphate, or a combination thereof. In some embodiments, the oral care composition may further include a humectant, such as sorbitol, glycerin, xylitol, polyethylene glycol, propylene glycol, or a combination thereof. In some embodiments, the oral care composition may include a bicarbonate salt such as sodium bicarbonate, potassium bicarbonate, ammonium bicarbonate, or a combination thereof. In some embodiments, the oral care composition may also include hydrogen peroxide for whitening of the teeth. In some embodiments, the oral care composition may include a foaming agent, such as a high molecular weight polyethylene glycol (PEG). In one aspect, rhamnolipids of the present technology can be formulated (or otherwise used) as natural foaming agents, having similar properties to synthetic foaming agents. For example, rhamnolipids of the present technology have similar foaming properties to sodium lauryl ether sulfate (SLES) in rinse-off applications (e.g., as indicated using standardized shake foam methods). Since the mechanism of foaming in oral care has significantly larger shear in a smaller, more confined area, the foaming profile of the rhamnolipids disclosed herein can translate to oral care as a natural foaming agent alternative. In some embodiments, the oral care composition may also include an antisensitivity agent.

In some embodiments, and in particular where the oral care composition is a mouthwash, the oral care composition may include an alcohol, such as ethanol.

In some embodiments, and in particular where the oral care composition is a toothpaste, the oral care composition may include a binder. Examples of suitable binders include carboxymethyl celluloses, hydroxyethyl celluloses, hydroxyethyl carboxymethyl celluloses, natural and synthetic gums, polyvinyl pyrrolidone, colloidal carboxyvinyl polymers, seaweed colloids, and the like.

In some embodiments, the oral care composition may include a flavoring agent, or flavorant. Suitable flavorants include oils of wintergreen, peppermint, and spearmint.

The oral care compositions of the present technology can have pH values in the range of from about 5 to about 12; alternatively, from about 6 to about 8. Solubility of the rhamnolipid component alone in water decreases as the pH is lowered from 6 to 5 as the rhamnolipid protonates.

The oral care compositions and/or formulations of the present technology can be in a variety of forms, such as an aqueous solution, gel, paste, spray, cream, homogeneous or non-homogeneous mixture, or a powder. End uses of the compositions include, but are not limited to, dentifrices, oral rinses, mouthwash, toothpaste, toothpowder, dissolvable strips, lozenges, chewing gum, wipes, or any other oral care product known in the art. The oral care compositions may be used to inhibit and/or reduce planktonic presence and/or biofilm (e.g. dental plaque) formation, and have shown inhibitory and bactericidal efficacy against oral bacteria, including P. gingivalis, S. mutans, and C. rectus.

The oral care composition can, in some embodiments, be used to treat and/or prevent plaque, biofilm, and/or tartar formation; gingivitis, tooth decay, periodontal disease, or any other oral health condition related to the presence or build-up of oral bacteria. The frequency of administration of the oral care composition may be configured depending on the specific type of product, the strength of the product (the dosage provided per use), the state of a user's oral health, and the like. In some embodiments, for example, the oral care composition can be administered once per day, twice per day, three time per day, once every two days, once every three days, or the like.

The oral care composition may be administered prophylactically, i.e. in order to prevent plaque, biofilm, and/or tartar formation, gingivitis, tooth decay, periodontal disease, or any other oral health condition related to the presence or build-up of oral bacteria in an individual, or as part of a treatment plan, i.e. to treat plaque, biofilm, and/or tartar formation, gingivitis, tooth decay, periodontal disease, or any other oral health condition related to the presence or build-up of oral bacteria in an individual suffering from one or more of those oral health conditions. For prophylactic treatment, the individual may not be suffering from the oral health condition that administration of the oral care composition is configured to prevent. In some embodiments, however, the individual may be particularly prone or susceptible to the oral health condition that administration of the oral care composition is configured to prevent.

When used to treat plaque, biofilm, and/or tartar formation, gingivitis, tooth decay, periodontal disease, or any other oral health condition related to the presence or build-up of oral bacteria in an individual suffering from one or more of those oral health conditions, the dosage regimen can continue for one, two, three, four, five, six, or more weeks according to the condition and response of the patient. At the end of the treatment, the oral care composition or a variant thereof (e.g. one containing a lower dosage of active) may continue to be used as a prophylactic treatment.

One skilled in the art will recognize that modifications may be made in the present technology without deviating from the spirit or scope of the invention. The invention is further illustrated by the following examples, which are not to be construed as limiting the invention in spirit or scope to the specific procedures or compositions described therein.

### EXAMPLES

### Example 1: P. gingivalis Inhibitory and Bactericidal Efficacy Testing

The Minimum Inhibitory Concentration (MIC) and the Minimum Bactericidal Concentration (MBC) were determined using rhamnolipid active-only compositions of the present disclosure, for the representative oral care relevant microorganism Porphyromonas gingivalis.

As used herein, the MIC is the concentration of rhamnolipid at which no growth of the target bacteria is detected after sufficient incubation in nutrient broth media against controls. The MIC was determined in accordance with industry standards Clinical and Laboratory Standards Institute (CLSI) M7, M11, and M26 in that samples were serially diluted, typically 1:2 or 1:3, in nutritive medium suitable for the growth of the tested microorganism and sufficiently pre-reduced to remove oxygen prior to conducting anaerobic testing. Pre-reduction was conducted by using an indirect atmospheric sequestering of oxygen using hydrogen enabled reaction over palladium catalyst. Subsequently, samples were exposed to a minimum of 1.0E+06 CFU/mL final concentration of the test challenge microorganism. Following an initial incubation period sufficient for positive controls (nutrient media, microorganisms and water instead of active ingredients) to develop mature and measurable growth, samples were evaluated for presence/absence of growth visually by turbidity assessments. Samples which did not turn turbid as compared to positive controls and were comparable to negative controls marked the concentration of inhibitory action.

As used herein, the MBC is the lowest concentration of the antibacterial composition required to kill a specific target bacteria. The MBC was determined in accordance with industry standards CLSI M7, M11, and M26. Test samples showing inhibitory effectiveness in the MIC test were further tested for bactericidal properties by neutralizing an aliquot extracted from each concentration tested and plated onto appropriate agar medium to determine presence of survivors. Samples which showed ≥3.0 Log 10 reduction (≥99.9% reduction) against controls were determined to be the bactericidal concentrations.

The representative strain tested was Porphyromonas gingivalis (Pg) ATCC 33277.

The representative Rhamnolipid compositions tested included:

| | | | Mono:Di |
|---|---|---|---|
| | | | Rhamnolipid |
| | | % Active | Ratio |
| Sample | Active Agent | Agent | (via HPLC) |
| Sample 1 (mixture of rhamnolipids obtained | Rhamnolipid | 30.7 | 44:56 |
| by solvent extraction from a concentrated | | | |
| clarified broth and treated with hydrogen | | | |
| peroxide) | | | |
| Sample 2 (mixture of rhamnolipids obtained | Rhamnolipid | 29.7 | 44:56 |
| by solvent-free extraction from a | | | |
| concentrated clarified broth) | | | |
| Sample 3 (mixture of rhamnolipids obtained | Rhamnolipid | 29.7 | 44:56 |
| by solvent extraction from a concentrated | | | |
| clarified broth) | | | |
| Comparative Sample | Rhamnolipid | 42.2 | 4:96 |
| (commercially available rhamnolipid | | | |
| product) | | | |

These Rhamnolipid samples, tested against Porphyromonas gingivalis (ATCC 33277), exhibited the Minimum Inhibitory Concentration (MIC) values, and Minimum Bactericidal Concentration (MBC) values shown in Table 1.

TABLE 1

| | |
|---|---|
| Rhamnolipid Active | |
| Level Test Range | Results (ppm) |

| Sample | (ppm) | MIC | MBC |
|---|---|---|---|
| 1 | 4.6-30,000 | 41 | 123 |
| 2 | 4.6-30,000 | 41 | 123 |
| 3 | 4.6-30,000 | 41 | Not Tested |
| Comparative | 4.6-30,000 | 132 | 370 |

Minimum Inhibitory Concentration (MIC) = No Growth Detected After ≥72 h
Minimum Bactericidal Concentration (MBC) = ≥99.9% Reduction After ≥22 h

The tested rhamnolipid mixture showed improved inhibitory and bactericidal effectiveness against P. gingivalis ATTC 33277 using the common microbiological methods referred to as Minimum Inhibitory Concentration (MIC) and Minimum Bactericidal Concentration (MBC).

### Example 2: S. mutans Inhibitory and Bactericidal Efficacy Testing

The MIC and MBC values, for a representative rhamnolipid composition of the present disclosure, were also determined against the oral care relevant microorganism Streptococcus mutans.

The representative strain tested was Streptococcus mutans ATCC 25175. The rhamnolipid composition tested was Sample 1 used above for Porphyromonas gingivalis. The comparative composition was the same Comparative Sample used above for Porphyromonas gingivalis.

As shown in Table 2, the rhamnolipid compositions of Sample 1 and the comparative sample showed MIC values of 123 ppm and 1111 ppm, respectively. The rhamnolipid compositions of Sample 1 and the comparative sample also showed MBC values of 1111 ppm and 3333 ppm, respectively. Both compositions were tested against Streptococcus mutans ATCC 25175.

TABLE 2

| | |
|---|---|
| Rhamnolipid Active | |
| Level Test Range | Results (ppm) |

| Sample | (ppm) | MIC | MBC |
|---|---|---|---|
| 1 | 4.6-30,000 | 123 | 1111 |
| Comparative | 4.6-30,000 | 1111 | 3333 |

Minimum Inhibitory Concentration (MIC) = No Growth Detected After ≥48 h
Minimum Bactericidal Concentration (MBC) = ≥99.9% Reduction After ≥48 h

The tested rhamnolipid mixture showed improved inhibitory and bactericidal effectiveness against S. mutans ATCC 25175 using the common microbiological methods referred to as Minimum Inhibitory Concentration (MIC) and Minimum Bactericidal Concentration (MBC).

### Example 3: C. rectus Inhibitory and Bactericidal Efficacy Testing

The MIC and MBC values, for a representative rhamnolipid composition of the present disclosure, was also determined against the oral care relevant microorganism Campylobacter rectus.

The representative strain tested was Campylobacter rectus ATCC 33238. The rhamnolipid composition tested was Sample 1 used above for Porphyromonas gingivalis. The comparative composition was the same Comparative Sample used above for the Porphyromonas gingivalis example.

As shown in Table 3, the rhamnolipid compositions of Sample 1 and the comparative sample showed MIC values of 41 ppm and 123 ppm, respectively. The rhamnolipid compositions of Sample 1 and the comparative sample also showed MBC values of 41 ppm and 123 ppm respectively. Both compositions were tested against 1.9E+07 CFU/mL of Campylobacter rectus (Cr) ATCC 33238 conducted in Modified Reinforced Clostridial Broth (RMCB) after 28 h of exposure.

TABLE 3

| | |
|---|---|
| Rhamnolipid Active | |
| Level Test Range | Results (ppm) |

| Sample | (ppm) | MIC | MBC |
|---|---|---|---|
| 1 | 4.6-30,000 | 41 | 41 |
| Comparative | 4.6-30,000 | 123 | 123 |

Minimum Inhibitory Concentration (MIC) = No Growth Detected After ≥48 h
Minimum Bactericidal Concentration (MBC) = ≥99.9% Reduction After ≥28 h

The tested rhamnolipid mixture showed improved inhibitory and bactericidal effectiveness against C. rectus ATCC 33238 using the common microbiological methods referred to as Minimum Inhibitory Concentration (MIC) and Minimum Bactericidal Concentration (MBC).

The above examples demonstrate that rhamonolipid mixtures of the present disclosure have inhibitory and bactericidal effects against oral care relevant microorganisms, including e.g. Porphyromonas gingivalis, Streptococcus mutans, and Campylobacter rectus. The above examples also demonstrate that, surprisingly, an increase in the percentage by weight mono-rhamnolipids, i.e. an increase in the ratio of mono-rhamnolipids to di-rhamnolipids above the 4:96 ratio found in the comparative commercially available rhamnolipid product, can produce improved inhibitory and/or bactericidal effects against oral care relevant microorganisms, including e.g. Porphyromonas gingivalis, Streptococcus mutans, and Campylobacter rectus.

### Example 4: P. gingivalis Biofilm Qualitative Dissociation Testing

This test method assesses the ability of the test compound(s) to prevent the growth and establishment of biofilm of P. gingivalis.

The representative strain tested was Porphyromonas gingivalis (Pg) ATCC 33277. The rhamnolipid composition tested was Sample 3 used above for Porphyromonas gingivalis in Table 1.

First, a 1:10 culture of established Porphyromonas gingivalis. in nutrient media was prepared. A 96-well microtiter plate was then aliquoted with 180 microliters of the test culture and 20 microliters of either a control of sterile, deionized water or one of the following:
i. a solution containing rhamnolipids at a 2.6% active level
ii. a solution containing rhamnolipids at a 1.6% active level
iii. a solution containing rhamnolipids at a 0.6% active level
   More specifically, four wells of the plate were filled with 180 microliters of the culture and 20 microliters of the 2.6% active solution, four wells of the plate were filled with 180 microliters of the culture and 20 microliters of the 1.6% active solution, and four wells of the plate were filled with 180 microliters of the culture and 20 microliters of the 0.6% active solution. Each set of four was provided with an adjacent set of four control wells, each of which contained 180 microliters of the culture and 20 microliters of sterile, deionized water.

A lid was placed on the microtiter plate and then the plate was incubated for about 72±2 hours at a temperature of about 37±2° C.

At the end of the incubation period, each well was stained with 0.4% Crystal Violet and the results were evaluated via the following coloring score:
0—colorless
1—very light coloring
2—moderately light coloring
3—medium coloring
4—moderately dark
5—dark coloring.

Based on a visual observation, it was concluded that the P. gingivalis growth with introduction of the active rhamnolipid mixtures produced an observation score of 2 (moderately light coloring), compared to the control observation score of 5 (dark coloring). Therefore, it was concluded that the introduction of the rhamnolipid mixture dissociated established P. gingivalis biofilm as measured by this qualitative method.

### Example 5: P. gingivalis Biofilm Quantitative Dissociation Testing

This test method assesses the ability of the test compound(s) to dissolve, remove and/or kill an already formed P. gingivalis biofilm.

The representative strain tested was Porphyromonas gingivalis (Pg) ATCC 33277.

The rhamnolipid composition product of 0.165% by weight and 0.325% by weight was evaluated to assess dissociation efficacy abilities against already formed P. gingivalis biofilm. The test was conducted against common oral care organisms at a set contact time applicable to industry usage. First, the test organism was grown for 3 days in supplemented Tryptic Soy Broth (ATCC medium 2722) then 270 ul of the grown culture was transferred into a sterile 96-well plate to age for 48±2 hours allowing the culture to form a mature biofilm on the walls of the well plate and achieve a high titer.

To begin testing, the medium was suctioned out of the inoculated wells being careful not to disturb the biofilm. 270 ul Sterile DI water was added to the evaluation wells and 30 ul sterile DI was added to the initial 270 ul sterile DI well for the selected contact time to assess for control to complete a 1:10 dilution. After exposure contact time elapsed, the entire contents were suctioned out being careful to still not disturb the biofilm present on the well walls. The wells were washed twice with sterile DI water to aide in the quenching of the active. The exposure process was repeated with each test product (0.165% by weight product and 0.325% by weight product) for evaluation. A 1:10 dilution was completed for exposure, 270 ul Sterile DI water was added to the inoculated wells and 30 ul test product was added to the initial sterile DI well for the selected contact time to assess for sample efficacy then washed twice for quenching.

After exposures and washes, the plate was dried in an oven at for approximately 30 minutes. 270 ul of 0.1% Crystal Violet was added to each evaluated well and rested for approximately 15 minutes. The plate was submerged in DI water then turned over and blotted on paper towel for a total of 3 independent times with the water being replaced between each submersion. The plate sat to air dry for approximately 10 minutes and then 270 ul of 33% glacial acetic acid was added to each treated well to resuspend the bound dye to the adherent cells. Absorbance at 590 nm OD was measured and % dissociation was measured against positive control absorbance values.

As shown in Table 4, 0.325% by weight rhamnolipid composition produced an effective dissociation against common oral care organism, P. gingivalis, after a 30 sec contact time. Rhamnolipid compositions produced comparable dissociation efficacy results.

TABLE 4

| | | Rhamnolipid Dissociation Efficacy against P. | |
|---|---|---|---|
| | | gingivalis after 30 sec Contact Time | |
| | | (% Dissociation) | |
| Sample | pH | 0.325% active | 0.165% active |
| 3 | 7.3 | 81% | 77% |
| Comparative | 5.9 | 91% | 72% |

### Example 6: Flavor Solubilization

The ability of the rhamnolipid mixture to solubilize an oil-based flavorant, such as a mint oil flavorant, was tested. The rhamnolipid composition tested was Sample 3 used above for Porphyromonas gingivalis.

Functionality was determined through a series of dilutions using the rhamnolipid mixture as a potential solubilizer component for flavor oils in the absence of synthetic or other solubilizers. The rhamnolipid composition was compared with polysorbate 20, which is a synthetic solubilizer and the industry standard for oil solubilization for flavors. Common mint flavors, such as Mint #244456 and Mint #244455 from Belle Aire Creations, were used as the flavor oils.

The rhamnolipid composition was added to each of the flavor oils in increasing amounts in order to determine the solubilizer component to flavor oil ratio at which a clear premix was obtained. Once a clear premix was obtained, that premix was then added to deionized water to ensure that the final solution was crystal clear.

It was found that polysorbate 20 had a solubilization ratio of 1:6 (flavor oil:polysorbate 20) and the rhamnolipid mixture had a solubilization ratio of 1:13 (flavor oil:rhamnolipid mixture). In both instances, the final solution of the premix in deionized water was crystal clear.

The 1:13 ratio obtained by the rhamnolipid mixture is viewed as being sufficient to consider the rhamnolipid mixture an acceptable solubilizer for an oil-based flavorant, such as a mint flavorant. This ability may be of particular importance in some embodiments or composition types because otherwise the composition would have a translucent to opaque appearance that may be aesthetically, and thus commercially, undesirable.

### Example 7: P. gingivalis Prevention and Bactericidal Time-to-Kill Efficacy Testing

Efficacy of representative rhamnolipid compositions, of the present disclosure, were evaluated against P. gingivalis. Tests were conducted at a set contact time applicable to end usage. The selected test organism was grown for 3 days in medium mentioned previously, then passage again for an additional 48±2 hours allowing the culture to mature and achieve a high titer.

To begin testing, the prepared microorganism titer was pipetted into a 96-well plate at 270 ul volume. 30 ul of sterile DI water was added to prepare control samples. For test sample, 30 uL was used instead. After contact time exposure, sufficient 1:10 dilutions and/or use of neutralizer were applied to sufficiently neutralize the actives. Further dilutions and staining with viability dyes (Syto 9 and Propidium Iodide) were performed and samples were analyzed by flow cytometry. Flow cytometry data was used to calculate log 10 reductions.

As illustrated in Tables 5, rhamnolipid compositions of the present disclosure (at concentrations of 0.1%, 0.05%, and 0.025% by weight rhamnolipid compositions of the present disclosure, and when formulated as a finished product (having 0.1%, 0.05%, and 0.025% by weight rhamnolipid, based on the total weight of the composition) demonstrated a greater reduction (at each concentration) of P. gingivalis titer (within a 30 second contact time), when compared to a comparative rhamnolipid composition (at the same concentrations and contact times), and comparable reduction against the tested national brand mouthwash sample (i.e., Crest Pro-Health Advanced Multi-Protection with Sodium Fluoride 0.02% Active Ingredient).

Formulated Product(s) B (FP-B) in Table 5 comprise Polyoxamer 407, Sodium Benzoate, Citric Acid and active (i.e., Rhamnolipid Sample 3). Formulated Product(s) C (FP-C) in Table 5 are controls, and comprise the same material as the corresponding FP-B, except for Rhamnolipid Sample 3 and Citric Acid.

To prepare each of the FP-B compositions tested below (Table 5), a first FP-B bulk composition was prepared comprising: 0.29% Polyoxamer 407 (% by weight, based on the total weight of the composition); 0.19% Sodium Benzoate (% by weight, based on the total weight of the composition); 0.06% Citric Acid (% by weight, based on the total weight of the composition); and 1.0% Rhamnolipid Sample 3 (% by weight, based on the total weight of the composition). To prepare the individual FP-B samples tested below, this FP-B bulk composition was then diluted 1/10 (for FP-B having 0.1% Active), 1/20 (for FP-B having 0.05% Active), and 1/40 (for FP-B having 0.025% Active).

To prepare each of the corresponding FP-C compositions tested below (Table 5), a FP-C bulk composition was prepared comprising: 0.30% Polyoxamer 407 (% by weight, based on the total weight of the composition); and 0.20% Sodium Benzoate (% by weight, based on the total weight of the composition). To prepare the individual FP-C samples tested in Table 6, this FP-C bulk composition was then diluted 1/10 (for FP-C control corresponding to FP-B having 0.1% Active), 1/20 (for FP-C control corresponding to FP-B having 0.05% Active), and 1/40 (for FP-C control corresponding to FP-B having 0.025% Active).

TABLE 5

| | | Rhamnolipid Prevention Efficacy against P. | | |
|---|---|---|---|---|
| | | gingivalis after a 30 sec Contact Time | | |
| | | (Log10 Reduction) | | |
| Sample | pH | 0.1% active | 0.05% active | 0.025% active |
| 3 | 7.3 | >5.89 | >5.89 | 3.37 |
| Comparative | 6.9 | 2.85 | 2.10 | 1.49 |
| Formulated Product-B (FP-B) | 6.5 | >5.89 | 4.01 | 2.54 |
| (Having Sample 3 | | | | |
| Rhamnolipid As Active) | | | | |

| Formulated Product/No Active | | 0.0% active | 0.0% active | 0.0% active |
|---|---|---|---|---|
| Formulated Product-C (FP-C) | 4.8 | 0.52 | 0.38 | 0.22 |
| (No Active) | | | | |

| Benchmark | | 0.018% active | 0.010% active |
|---|---|---|---|
| Crest Pro-Health Advanced | 3.4 | >5.89 | 4.30 |
| Multi-Protection | | | |

The embodiments and examples described here are illustrative, and do not limit the presently described technology in any way. The scope of the present technology described in this specification is the full scope defined or implied by the claims. Additionally, any references noted in the detailed description section of the instant application are hereby incorporated by reference in their entireties, unless otherwise noted.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further the examples are provided to not be exhaustive but illustrative of several embodiments that fall within the scope of the claims.

What is claimed is:

1. An oral care composition comprising:
(a) a mixture of rhamnolipids in an amount of 0.025% to 99% by weight, based on the total weight of the composition,
wherein the mixture of rhamnolipids comprises mono-rhamnolipids and di-rhamnolipids in a weight ratio of 40:60 to 60:40 mono-rhamnolipids:di-rhamnolipids,
wherein Rha-C10-C10 mono-rhamnolipid is about 5% to about 38% by weight and RhaRha-C10-C10 di-rhamnolipid is about 34% to about 45% by weight based on the total weight of the mixture of rhamnolipids; and
(b) at least one orally acceptable carrier, and optionally one or more additives, in an amount to total 100% by weight of the composition.

2. The oral care composition of claim 1, wherein the composition comprises an amount of RhaRha-C10-C12 di-rhamnolipid of about 5% to about 15%, preferably about 10% to about 12.5% by weight based on the total weight of the mixture of rhamnolipids.

3. The oral care composition of claim 1, wherein the composition comprises an amount of RhaRha-C10-C12:1 di-rhamnolipid of about 0.2% to about 5%, preferably about 1% to about 4% by weight based on the total weight of the mixture of rhamnolipids.

4. The oral care composition of claim 1, wherein the composition comprises an amount of RhaRha-C8-C10 di-rhamnolipid of about 0.2% to about 5%, preferably about 1% to about 4% by weight based on the total weight of the mixture of rhamnolipids.

5. The oral care composition of claim 1, wherein the composition comprises an amount of Rha-C10-C12 mono-rhamnolipid of about 0.2% to about 6%, preferably about 3.5% to about 5% by weight based on the total weight of the mixture of rhamnolipids.

6. The oral care composition of claim 1, wherein the composition comprises an amount of Rha-C8-C10 mono-rhamnolipid of about 0.2% to about 5%, preferably about 1% to about 4% by weight based on the total weight of the mixture of rhamnolipids.

7. The oral care composition of claim 1, wherein the one or more additives are present in the composition and comprise one or more of surfactants, thickeners, preservatives, flavorants, colorants, humectants, buffers, chelating agents, sweeteners, fluoride sources, and abrasive agents.

8. The oral care composition of claim 1, wherein the orally acceptable carrier comprises water.

9. The oral care composition of claim 1, wherein the composition exhibits an inhibitory effect against P. gingivalis, having a minimum inhibitory concentration (MIC) of from about 21 ppm to about 106 ppm.

10. The oral care composition of claim 1, wherein the composition exhibits a bactericidal effect against P. gingivalis, having a minimum bactericidal concentration (MBC) of from about 62 ppm to about 296 ppm.

11. The oral care composition of claim 1, wherein the composition exhibits an inhibitory effect against S. mutans, having a minimum inhibitory concentration (MIC) of from about 62 ppm to about 889 ppm.

12. The oral care composition of claim 1, wherein the composition exhibits a bactericidal effect against S. mutans, having a minimum bactericidal concentration (MBC) of from about 556 ppm to about 2666 ppm.

13. The oral care composition of claim 1, wherein the composition exhibits an inhibitory effect against C. rectus, having a minimum inhibitory concentration (MIC) of from about 21 ppm to about 98 ppm.

14. The oral care composition of claim 1, wherein the composition exhibits a bactericidal effect against C. rectus having a minimum bactericidal concentration (MBC) of from about 21 ppm to about 98 ppm.

15. The oral care composition of claim 1, wherein the composition exhibits a P. gingivalis Log₁₀ reduction of 3.0 to 6, with rhamnolipid present in an amount of 0.1% by weight of the composition at a contact time of 30 seconds.

16. The oral care composition of claim 1, wherein the composition exhibits a P. gingivalis Log₁₀ reduction of 2.5 to 6, with rhamnolipid present in an amount of 0.05% by weight of the composition at a contact time of 30 seconds.

17. The oral care composition of claim 1, wherein the composition exhibits a P. gingivalis Log₁₀ reduction of 2.0 to 6, with rhamnolipid present in an amount of 0.025% by weight of the composition at a contact time of 30 seconds.

18. The oral care composition of claim 1, wherein the composition has a pH of 6.0 to 8.0.

19. The oral care composition of claim 1, wherein the composition contains a flavorant selected from the following: oils of wintergreen mint, spearmint, peppermint, or any combination thereof and wherein the mixture of rhamnolipids is present in an amount effective to solubilize the flavorant.

20. A method of treating or preventing gingivitis in a subject afflicted with or susceptible to gingivitis comprising administering to the subject an effective amount of the oral care composition of claim 1, thereby treating or preventing gingivitis in the subject.
